(19) United States
(12) Patent Application Publication
(10) Pub. No.: US 2024/0415697 A1
(43) Pub. Date: Dec. 19, 2024

(54) DEVICE AND SYSTEM FOR LIQUID DELIVERY TO A SURFACE OF AN EYE

(71) Applicant: Topnote, Inc., Newport Coast, CA (US)

(72) Inventor: Ehud Ivri, Newport Coast, CA (US)

(21) Appl. No.: 18/739,948

(22) Filed: Jun. 11, 2024

Related U.S. Application Data
(60) Provisional application No. 63/628,075, filed on Jun. 17, 2023.

(51) Int. Cl. A61F 9/00; A61B 17/00; A61M 39/24; B05B 5/025

(52) U.S. Cl. CPC A61F 9/0026; A61M 39/24; B05B 5/025; A61B 2017/00132; A61B 2017/00398; A61B 2017/00477; A61B 2017/00734

(57) ABSTRACT

Methods, systems, devices, and apparatuses for dispensing a predetermined amount of fluid to a surface of an eye. A dispensing system includes one or more bottle assemblies each including a bottle enclosure and a cup-shaped member coupled to the bottle enclosure to form a hermetically sealed fluid compartment within the bottle enclosure for holding the fluid. The one or more bottle assemblies further include a dispensing mechanism coupled to the cup-shaped member and configured to dispense the predetermined amount of the fluid. The dispensing system further includes a reusable electric dispensing actuator configured to removably couple to a bottle assembly of the one or more bottle assemblies by sliding into the cup-shaped member and to activate the dispensing mechanism to pull the fluid from the hermetically sealed fluid compartment and dispense the fluid through a dispensing tip.

### CROSS-REFERENCE TO RELATED APPLICATIONS

This application claims priority to and the benefit of U.S. Provisional Patent Application No. 63/628,075 filed on Jun. 17, 2023, the entirety of which is hereby incorporated by reference.

### BACKGROUND

### 1. Field

This specification relates to systems, devices, and/or methods for dispensing an amount of fluid or liquid in one or more discrete drops onto a surface of an eye, more specifically, the device being capable of generating the one or more discrete drops for the optimal and convenient administration of a predetermined dose of fluid.

### 2. Description of the Related Art

Conventional methods of ocular administration of an aqueous solution (e.g., an eyedrop) generally utilizes squeeze bottle dispensers. Administration of the eyedrop generally requires that a recipient tilt his head (e.g., from a vertical position toward a horizontal position) which leads to inefficiency, discomfort, and uncertainty over an amount of the eyedrop that actually reaches the target (e.g., an eye of the recipient).

Administration of eyedrops using squeeze bottle dispensers also produces a large drop of liquid to the eye that initiates a blink reflex, which can result in a substantial wastage of the applied liquid drug and drainage either through tear ducts and/or onto the skin surface. Certain devices attempt to overcome this problem by using an electronically controlled dispensing system which generates a stream of droplets with a certain dose volume. U.S. Pat. No. 9,801,757, U.S. Pat. Pub. No. 2014/0336618A1, and U.S. Pat. No. 11,011,270 describe a dispensing device that produces a jet or mist to the eye using a piezoelectric or an electromechanical dispensing system. These systems include electronic control circuits and batteries that make the device larger and less portable compared to pocketsize squeeze bottle dispensers that typically comprise a small bottle with a volume of 10-30 milliliters (mL). Size and portability are particularly important for patients that need several treatments throughout the day whereby pocketsize devices are preferred. In addition, prior devices may be expensive due to having to replace the whole device when depleted.

Accordingly, there is a need for a system, a device, and/or a method for dispensing an amount of fluid or liquid in one or more discrete drops onto a surface of an eye.

### SUMMARY

Disclosed herein is a system or device that utilizes an electromechanical dispensing system but keeping a form factor, size, and shape of the device substantially similar to a small 10 milliliters (mL) or 15 milliliters (mL) squeeze bottle dispenser therefore having the benefit of both portability and ease of use and still having the same number of dose treatments in the device. Moreover, the electromechanical dispensing system is reusable and a fluid container can be removed and replaced without compromising the sterility of the system therefore further providing a cost-effective solution.

Disclosed herein is a delivery device for dispensing a small volume of liquid solution or suspension to a surface of an eye. The delivery device may have substantially the same size and form factor and the same number of treatment doses as a small squeeze bottle dispenser but the delivery device may further include an electronically controlled dispensing actuator which enables convenient delivery of a micro-dose without the delivery device having a large size and/or volume and without requiring inconvenient head tilting.

It is known that the volume of an eyedrop dispensed from a squeeze bottle dispenser is much larger than the volume that is retained to the surface of the eye. For example, U.S. Pat. No. 5,630,793 indicates that when an eyedrop of 30-50 microliters (μL) is applied to an eye, the actual amount that remained at the target is only 5-7 microliters (μL) which indicates that less than ⅓rd of the liquid volume effectively stays on the eye while more than ⅔rd of the liquid volume is wasted. However, the delivery device of the present application may use the electronically controlled dispensing system that effectively delivers a micro-dose volume of 10 microliters (μL) and therefore, the total fluid volume stored in the bottle is reduced to ⅓rd of the amount used by a standard squeeze bottle dispenser, while the number of dose treatments stay the same.

The delivery device utilizes the remaining ⅔rd of the bottle volume to place a compartment for holding a micro-dose dispensing actuator, its batteries and electronic circuit. As a result, the form factor, size and shape of the delivery device remain substantially the same as a small squeeze bottle dispenser but is also effectively sufficient to hold both the micro-dose dispensing actuator and a volume of liquid that has the same number of dose treatments as a similar sized squeeze bottle dispenser. The delivery device eliminates the need to repackage a dispensing system, electronic circuit, and batteries in a special enclosure as described, for example, in U.S. Pat. Nos. 11,011,270 and 8,684,980.

Advantageously, the dispensing actuator is reusable such that an empty bottle can be removed and replaced with a prefilled, hermetically sealed bottle without the risk of cross contamination therefore further providing an economical and cost-effective solution.

In examples, a device for dispensing a predetermined amount of fluid to a surface of an eye is disclosed. The device may comprise a bottle assembly. The bottle assembly may include a bottle enclosure having an opening and configured to hold the fluid. The bottle assembly may include a cup-shaped member that extends into the bottle enclosure through the opening of the bottle enclosure dividing an internal space of the bottle enclosure into a first compartment and a second compartment, the first compartment being a hermetically sealed fluid compartment defined by a volume between an internal surface of the bottle enclosure and the cup-shaped member, the second compartment being an internal volume inside the cup-shaped member. The bottle assembly may include a dispensing mechanism coupled to the cup-shaped member and configured to dispense the predetermined amount of the fluid. The device may include an electric dispensing actuator. The actuator may be configured to removably couple to the bottle assembly by sliding into the second compartment, and engage and activate the dispensing mechanism to pull the fluid from the first compartment and dispense the fluid through a dispensing tip.

In examples, a dispensing mechanism for ophthalmic delivery of fluid medicament to a surface of an eye is disclosed. The dispensing mechanism may comprise a hemispherical cavity sealed by a diaphragm and including: an inlet conduit in fluid communication with a fluid compartment, and an outlet conduit in fluid communication with a one-way valve. The dispensing mechanism may include a ball member that is concentrically aligned to the hemispherical cavity and tangentially engaged with the diaphragm and configured to cyclically displace the diaphragm into the hemispherical cavity.

In examples, a dispensing system for dispensing a predetermined amount of fluid to a surface of an eye is disclosed. The system may comprise a plurality of bottle assemblies. Each of the plurality of bottle assemblies may include a bottle enclosure having an opening. Each of the plurality of bottle assemblies may include a cup-shaped member configured to extend into the bottle enclosure through the opening of the bottle enclosure to form a hermetically sealed fluid compartment within the bottle enclosure for holding the fluid. Each of the plurality of bottle assemblies may include a dispensing mechanism coupled to the cup-shaped member and configured to dispense the predetermined amount of the fluid. The system may include an electric dispensing actuator configured to removably couple to a bottle assembly of the plurality of bottle assemblies by sliding into the cup-shaped member. The actuator may be configured to engage and activate the dispensing mechanism to pull the fluid from the hermetically sealed fluid compartment and dispense the fluid through a dispensing tip. The actuator may be configured to removably couple to another bottle assembly of the plurality of bottle assemblies when the fluid within the bottle assembly is depleted.

### BRIEF DESCRIPTION OF THE DRAWINGS

Other systems, methods, features, and advantages of the present disclosure will be apparent to one skilled in the art upon examination of the following figures and detailed description. Component parts shown in the drawings are not necessarily to scale and may be exaggerated to better illustrate the important features of the present disclosure. In the drawings, like reference numerals designate like parts throughout the different views.

FIG. 1A and FIG. 1B illustrate a side view of an example dispensing system relative to a 10 milliliter (mL) (0.33 fluid ounces (fl oz)) squeeze bottle dispenser, respectively.

FIG. 2A and FIG. 2B illustrate a perspective view and an exploded view of a bottle assembly of the example dispensing system of FIG. 1A, respectively.

FIG. 3A and FIG. 3B illustrate the bottle assembly of FIG. 2A with a dispensing actuator pulled out from the bottle assembly and inserted into bottle assembly, respectively.

FIG. 4A and FIG. 4B illustrate sectional views of the example dispensing system of FIG. 1A showing a diaphragm in a first dispensing state and a second dispensing state, respectively.

FIG. 5A illustrates a perspective view of an example dispensing system.

FIG. 5B illustrates a perspective sectional view of the example dispensing system of FIG. 5A showing an eccentric wheel.

FIG. 5C illustrates a perspective view of a bottle assembly of the example dispensing system of FIG. 5A separated from a dispensing actuator of the example dispensing system of FIG. 5A.

FIG. 6A illustrates an example dispensing tip (or nozzle) assembly having a one-way valve.

FIG. 6B illustrates the dispensing tip assembly of FIG. 6A assembled in a housing.

FIG. 6C illustrates the dispensing tip assembly of FIG. 6B installed on a bottle assembly.

### DETAILED DESCRIPTION

Disclosed herein are systems, apparatuses, devices, and methods for dispensing an amount of fluid or liquid in one or more discrete drops onto a surface of an eye. A dispensing system may include an electromechanical dispensing device for delivery of a fluid (e.g., a fluid medicament) to a surface of an eye (e.g., an eye of a user). The dispensing system may further include a bottle assembly and/or one or more replacement bottle assemblies. The electromechanical dispensing device may be coupled to the bottle assembly and/or may be configured to removably couple to the bottle assembly. The bottle assembly may be freestanding. In examples, the bottle assembly may be similar in size and/or shape to a 10 milliliter (mL) squeeze bottle dispenser.

The electromechanical dispensing device may further include an electromechanical micro-dose dispensing actuator and a horizontal dispensing tip. The electromechanical dispensing device may deliver a micro-dose of about 10 microliters (μL) that is known to have the same therapeutic effectiveness as a 30-50 milliliter (mL) dose produce by a squeeze bottle dispenser. Delivery of the micro-dose volume proportionally reduces the total fluid volume required to achieve the same amount of doses as, for example, a 10 milliliter (mL) squeeze bottle dispenser and thus provides for an amount of saved space within the bottle assembly. The electromechanical dispensing device utilizes the saved space for a compartment for a reusable micro-dose dispensing actuator, in this way the electromechanical dispensing device has substantially the same form-factor, size, and/or shape as a squeeze bottle dispenser but further includes the reusable micro-dose dispensing actuator that conveniently delivers a smaller dose while enabling a recipient to face forward instead of having to tilt their head.

Moreover, the dispensing system and/or the electromechanical dispensing device provides a cost-effective solution without the risk of cross contamination by having the micro-dose dispensing actuator be reusable while the bottle assembly may be disposed and replaced with a new prefilled hermetically sealed bottle assembly. Moreover, the dispensing system may include a plurality of prefilled hermetically sealed bottle assemblies.

In the present disclosure, various terms may be used for which the following definitions will apply: “Jet dispensing” as used herein and sometimes referred to as “dispensing,” refers to a non-contact administration process that utilizes a fluid jet to form one or more droplets of liquid and expel them from a dispensing tip or a nozzle. The foregoing terms were also used in U.S. Pat. No. 9,039,666 entitled “Method and Apparatus for Liquid Dispensing” which is incorporated herein by reference for all purposes.

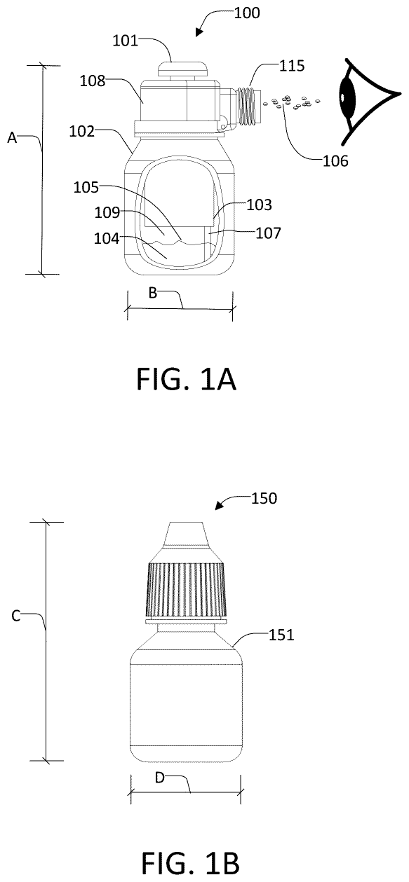

FIG. 1A and FIG. 1B illustrate a dispensing system 100 (also may be referred to as a dispensing device 100) relative to a conventional squeeze bottle dispenser 150 (e.g., a 10 milliliter (mL) (0.33 fluid ounces (fl oz)) squeeze bottle dispenser). Both the dispensing system 100 and the conventional squeeze bottle have substantially the same physical size and volume. The dispensing system 100 may include a bottle (or bottle enclosure) 102 and a dispensing actuator (or micro-dose dispensing actuator assembly) (or electric dispensing actuator) 108. For clarity, FIG. 1A illustrates the bottle 102 with a cut-out to show internal components of the bottle 102. In examples, the bottle 102 may have a cylindrical shape. In examples, the bottle 102 may have an internal volume of about 10 milliliters (mL). The dispensing actuator 108 may be configured to deliver a micro-dose of fluid (e.g., about 10 microliters (μL)) which is known to have the same therapeutic effectiveness as a 30 microliters (μL) dose generally produced by conventional squeeze bottle dispensers 150. In this way, the dispensing system 100 may require ⅓rd of the fluid volume and correspondingly ⅓rd of the internal volume relative to the squeeze bottle dispenser 150 for the same number of doses of fluid.

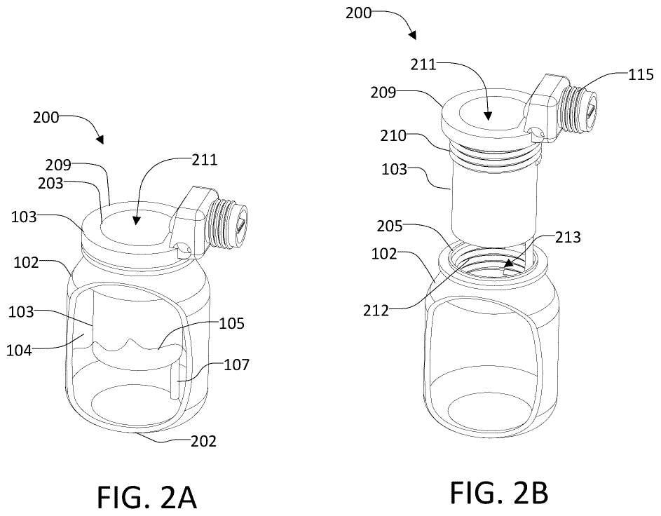

The dispensing system 100 may further include a cup-shaped member 103. At least a portion of the remaining ⅔rd of the internal volume of the bottle 102 (i.e., the internal volume of the bottle 102 that is not used to hold fluid) may provide an enclosure for the cup-shaped member 103. The cup-shaped member 103 may provide an enclosure for the dispensing actuator 108. In examples, the cup-shaped member 103 may have a cylindrical shape. The cup-shaped member 103 may extend into the bottle 102 through an opening 213 (not yet shown, marked in FIG. 2B) of the bottle 102 thereby dividing the internal volume of the bottle 102 into a fluid compartment (or first volume or subspace) 104 and a dispensing actuator compartment (or second volume or subspace) 211 (not yet shown, marked in FIG. 2A). The fluid compartment 104 may be a hermetically sealed space with a fluid 105 volume of, for example, 3.33 milliliters (mL) between an internal wall 109 of the bottle 102 and the cup-shaped member 103. The fluid compartment 104 may be defined by a volume between an internal surface of the bottle 102 and the cup-shaped member 103. The fluid compartment 104 may define the hermetically sealed enclosure that is used for storing fluid 105 (e.g., 3.33 milliliters (mL) of eyedrop medicament). In examples, the dispensing system 100 may have a fluid compartment 104 volume of about 3.33 milliliters (mL) and may be able to deliver about 333 doses of about 10 microliters (μL) of fluid 105 each dose. In this way the dispensing system 100 enables convenient treatment with the same number of doses available from the 10 milliliters (mL) squeeze bottle dispenser 150 that delivers about 333 doses of about 30 microliters (μL) of fluid each dose. In examples, the dispensing system 100 may be configured to dispense a predetermined amount of fluid upon each actuation of the dispensing system 100 through a dispensing end 115. For example, the predetermined amount of fluid may be about 5-20 microliters (μL) or less than 30 microliters (μL).

The dispensing system 100 may have a height A and a width B. In examples, the height A of the dispensing system 100 may be about 20 millimeters (mm) to about 60 millimeters (mm). In examples, the height A of the dispensing system 100 may be about 45 millimeters (mm). The squeeze bottle dispenser 150 may have a height C of about 55 millimeters (mm) and a width D of about 25 millimeters (mm). Thus, the dispensing system 100 may deliver the same amount of doses or more doses than the squeeze bottle dispenser 150 while being similar or smaller in size than the squeeze bottle dispenser 150. The bottle 102 may be similar in size to a 10 milliliters (mL) bottle 151 of the squeeze bottle dispenser 150 illustrated in FIG. 1B. In examples, the dispensing system 100 may include a larger bottle 102 such that the bottle 102 may hold more than 3.33 milliliters (mL) of fluid 105 (e.g., 5-10 milliliters (mL)). In examples, the dispensing system 100 may include a bottle 102 having the form factor and/or volume of conventional eyedrop dispensers having, for example, volumes of 10-30 milliliters (mL).

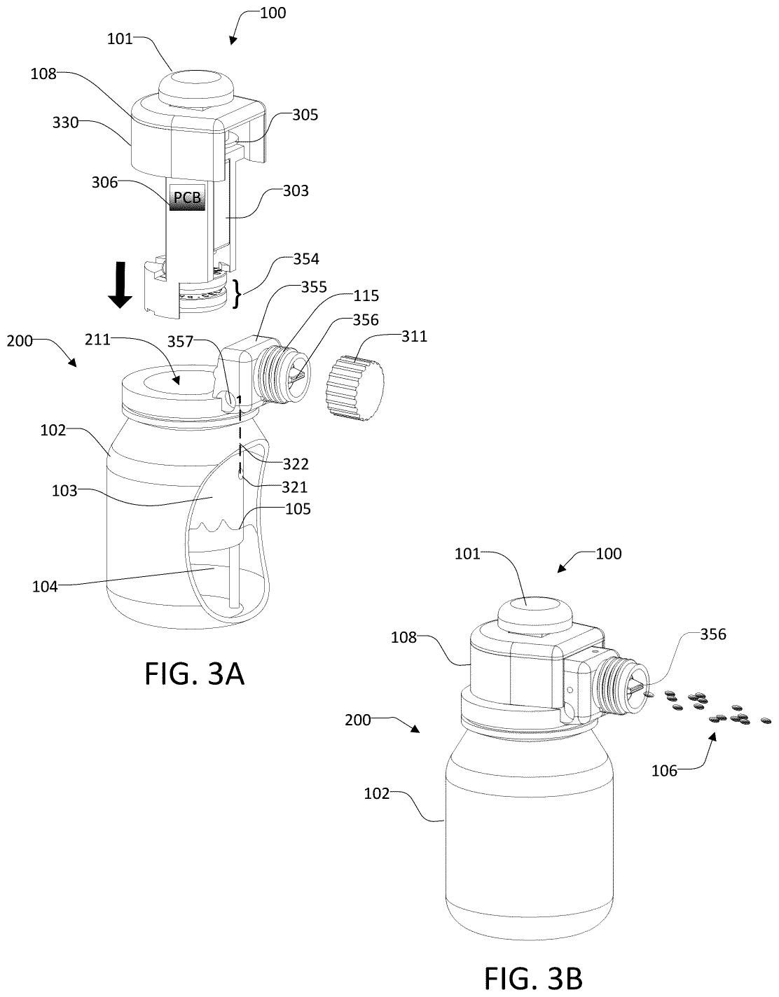

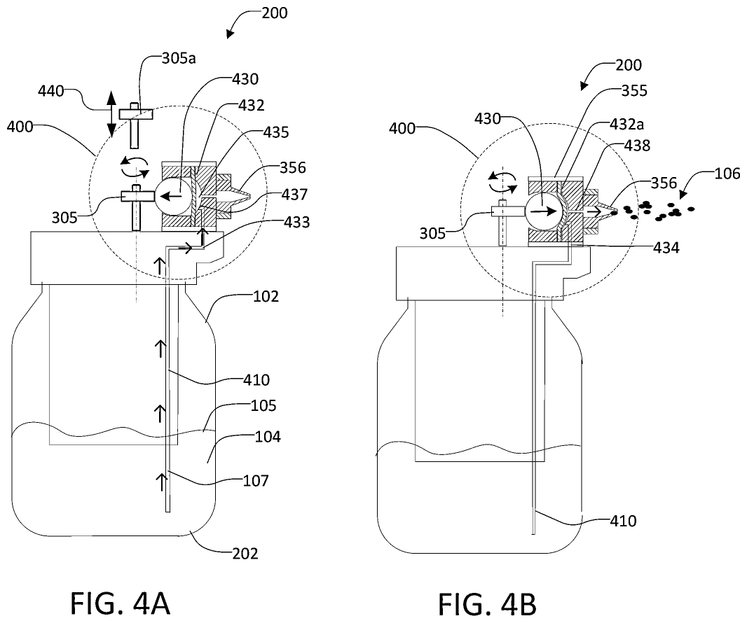

The dispensing system 100 may further include the dispensing end 115, a dip tube 107, and/or a conduit 433 (not yet shown, marked in FIG. 4A) between the dispensing end 115 and the dip tube 107. The dip tube 107 and the conduit 433 may deliver the fluid 105 to the dispensing end 115 to dispense droplets 106 when a momentary switch 101 is activated (e.g., by being pressed by a user). In examples, the dispensing end 115 may include one or more threads configured to receive a threaded cap 311 (not yet shown, marked in FIG. 3A).

FIG. 2A and FIG. 2B illustrate a bottle assembly 200 and an exploded view of the bottle assembly 200, respectively. The bottle assembly 200 may include the bottle 102 and the cup-shaped member 103. FIG. 2A illustrates the cup-shaped member 103 fully assembled to the bottle 102. As discussed above, the cup-shaped member 103 may divide the internal volume of the bottle 102 into two compartments including the fluid compartment 104 and the dispensing actuator compartment 211. The fluid compartment 104 may be a hermetically sealed compartment within the bottle 102 and outside the cup-shaped member 103. The fluid compartment 104 may be used for storing the fluid 105 (e.g., liquid medicament).

The dispensing actuator compartment 211 may be an open space within the cup-shaped member 103 such that the cup-shaped member 103 defines the dispensing actuator compartment 211. The dispensing actuator compartment 211 may be an internal volume within the cup-shaped member 103. In examples, the dispensing actuator compartment 211 may have a volume of about 6 milliliters (mL). The dispensing actuator compartment 211 may be configured for storing a removable dispensing actuator 108 (or electric dispensing actuator) including an electronic circuit and/or one or more batteries of the dispensing actuator 108. The dispensing actuator 108 may be configured to removably couple to the bottle 102 by sliding into the dispensing actuator compartment 211 and engage and activate the dispensing mechanism (or dispensing assembly) 400 to pull the fluid 105 from the fluid compartment 104 and dispense the fluid 105 through a dispensing tip (or one-way valve or horizontal valve) 356.

The dispensing actuator compartment 211 may be configured to store and/or removably couple to the dispensing actuator 108. By having the dispensing actuator 108 be removable, the dispensing system 100 maintains the form-factor, shape and/or size of a standard squeeze bottle dispenser 150 but has the further benefit of having the dispensing actuator 108 be reusable after removing the dispensing actuator 108 from the bottle assembly 200. The bottle assembly 200 provides a cost-effective enclosure for both the fluid 105 and the dispensing actuator 108. The dispensing actuator 108, which may be relatively heavy, is stored within the cup-shaped member 103 that extends into bottle 102 therefore making the center of mass of the dispensing system 100 closer to a bottom 202 of the bottle 102 such that the bottle 102 remains stable and free-standing.

The cup-shaped member 103 may further include a flange 209 around an opening 203 of the cup-shaped member 103. When assembled, in examples, the cup-shaped member 103 may be fastened by thread engagement such that the flange 209 is seated tightly against a sealing lip 205 that protrudes around the opening 213 of the bottle 102 creating the hermetically sealed fluid compartment 104. For example, the cup-shaped member 103 may include one or more threads 210 that are configured to thread into one or more threads 212 of the bottle 102. Alternatively, in examples, the cup-shaped member 103 may be coupled to a bottle 102 by an interference fit with a snap lock or any means (e.g., an adhesive, thermal bonding, etc.) that creates a hermetically sealed fluid enclosure or compartment within the bottle 102.

FIG. 3A illustrates an exploded view of the dispensing system 100 which includes the dispensing actuator 108 and the bottle assembly 200. The cup-shaped member 103 may further include a nozzle block 355. The nozzle block 355 may include the dispensing end 115, a dispensing tip (or one-way valve or horizontal valve) 356, and/or a venting port 357 that extends from the fluid compartment 104 to an air filter that is open to the atmosphere. The venting port 357 may equalize the pressure inside the fluid compartment 104 with the atmospheric pressure. In examples, the venting port 357 may be similar in function to a venting system that is described in U.S. Pat. No. 9,238,532 or U.S. Pat. Pub. No. 2014/0336596. Both patents are incorporated herein by reference for all purposes. The venting port 357 may include, for example, a 0.2 micrometer (μm) air filter that is capable of removing particles and microorganisms thereby preventing the particles and microorganisms from contaminating the fluid compartment 104. In examples, the venting port 357 may be connected to the fluid compartment 104 via a conduit (or vent path) 322 and/or an opening 321 in the cup-shaped member 103.

The dispensing actuator 108 may include an actuator housing 330. The dispensing actuator 108 and/or the actuator housing 330 may include an electric motor 303 (e.g., a DC motor) coupled to an eccentric wheel 305. The dispensing actuator 108 may further include one or more batteries 354 (e.g., one or two coin-cell batteries, one or more rechargeable batteries, etc.) that are electrically connected to the motor 303 and may be located at least partially within the actuator housing 330. The dispensing actuator 108 may further include the momentary switch 101. The momentary switch 101 may be configured to be finger actuated. The dispensing actuator 108 may further include a printed circuit board (PCB) (or electronic circuit) 306 electrically connected to the motor 303, the one or more batteries 354, and/or the momentary switch 101. The PCB 306 may be located at least partially within the actuator housing 330. The PCB 306 may include a timer circuit configured to control an activation, a duration, and/or a number of rotations of the motor 303. In examples, when the momentary switch 101 is actuated, the PCB 306 may initiate rotation of the eccentric wheel 305 by the motor 303 for a period of 50-100 milliseconds (msec) when actuated by a user. In the preferred embodiment the dispensing actuator 108 delivers a dose of about 10 microliters (μL) in less than about 100 milliseconds (ms).

The dispensing actuator 108 may be inserted into the dispensing actuator compartment 211 by, for example, a user and be mechanically engaged with the bottle assembly 200 to dispense the fluid 105. When inserted, the dispensing actuator 108 does not make contact with the fluid 105 within the hermetically sealed bottle assembly 200. The dispensing actuator 108 can be removed from an empty bottle assembly and inserted into a second pre-filled bottle assembly without the risk of cross contamination.

FIG. 3B illustrates the dispensing system 100 with the dispensing actuator 108 fully inserted into the dispensing actuator compartment 211 within the bottle assembly 200. Upon activation of the momentary switch 101, a stream of droplets 106 may be dispensed from the dispensing tip 356.

FIG. 4A and FIG. 4B illustrate a sectional view of the bottle assembly 200 which includes a dispensing mechanism (or dispensing assembly) 400 that is configured to pull the fluid 105 from the bottom 202 of the bottle 102 up to the dispensing tip 356 to dispense the fluid 105. In examples, the dispensing mechanism 400 may be within or at least partially within the nozzle block 355. The dispensing mechanism 400 may prime a fluid path (or passageway) 410 by pulling air and filling the fluid path 410 with the fluid 105. The fluid path 410 may include the dip tube 107 and/or the conduit 433. The dispensing mechanism 400 may further include a means to allow removal and insertion of the eccentric wheel (or dispensing actuator wheel) 305 by a sliding engagement such that an empty bottle assembly 200 can be readily replaced.

The dispensing mechanism 400 may include a solid structure with a hemispherical or substantially hemispherical cavity (or pump cavity) 435 sealed and/or covered by a diaphragm (or flat diaphragm) 432. The hemispherical cavity 435 may have a hemispherical or substantially hemispherical surface 437 at an end of the hemispherical cavity 435. The dispensing mechanism 400 may further include a ball member 430 that is tangentially engaged at a center of the diaphragm 432 on one side of the ball member 430 and with the eccentric wheel 305 on the opposite side of the ball member 430. The ball member 430 may be concentrically aligned to the hemispherical cavity 435 and tangentially engaged with the diaphragm 432. Rotation of the eccentric wheel 305 by the motor 303 cyclically displaces or oscillates the ball member 430 into the cavity 435 and/or toward the hemispherical surface 437 by deforming the diaphragm 432 from a planar or flat shape shown in FIG. 4A to a hemispherical or substantially hemispherical shape 432a shown in FIG. 4B. The dispensing actuator 108 activates the dispensing mechanism 400 by turning the eccentric wheel 305 to cyclically oscillate the ball member 430 and the diaphragm 432 toward the hemispherical surface 437 of the hemispherical cavity 435. Referring to FIG. 4B, it can be seen that the diaphragm 432 having the hemispherical shape 432a may have substantially the same radius of curvature as the cavity 435. In this way, displacement of the ball member 430 reduces the volume of the cavity 435 to zero or near zero, accordingly, the pressure and under-pressure induced by the displacement of the diaphragm 432 is maximized and sufficient to prime the fluid path 410. This principle is based on Boyle's Law wherein the pressure change in the cavity 435 is inversely proportional to the change in the cavity 435 volume. In examples, the volume of the cavity 435 shown in FIG. 4A may be about 8 cubic millimeters (mm\hat{ }3), this volume is reduced to zero or near zero therefore the volume ratio is maximized and sufficient to produce suction or under-pressure to prime the fluid path 410.

Referring to FIG. 4A, it can be seen that the cavity 435 is connected to the fluid path (or inlet conduit) 410. The cavity 435 may be in fluid communication with the fluid compartment 104 via the fluid path 410. In addition, the cavity 435 may be in fluid communication with the dispensing tip 356 via an outlet conduit 438. In this way, under-pressure that is generated in the cavity 435 pulls the fluid 105 from the bottle 102 into the cavity 435, while displacement of the diaphragm 432 into the cavity 435, as illustrated in FIG. 4B, displaces the fluid 105 through the dispensing tip 356 which emits a dispersion of droplets (or liquid particles) 106. The dispensing tip 356 may be and/or function as a one-way valve. Displacement of the diaphragm 432 into the cavity 435 may also generate a flow into the fluid path 410, however, a flow resistance through the dispensing tip 356 is lower than the flow resistance through the fluid path 410 and therefore the fluid 105 flows and is dispensed mostly through the dispensing tip 356 and minimally through the fluid path 410. In examples, the fluid path 410 may include a second one-way valve 434 that will completely stop fluid flow into the fluid path 410. Fluid 105 that is displaced through the dispensing tip 356 creates under-pressure within the fluid path 410 which pulls more fluid 105 from the bottle 102.

In a preferred embodiment a radius of the ball member 430 is about 2.5 millimeters (mm) and a thickness of the diaphragm 432 is about 0.5 millimeters (mm), accordingly, a radius of the deformed diaphragm 432a illustrated in FIG. 4B is about 3.0 millimeters (mm) which is the same as a radius of the hemispherical cavity 435. In examples, the displacement of the ball member 430 is about 0.7 millimeters (mm).

Referring to FIGS. 3A, 4A, and 4B, the eccentric wheel 305 may be included in the reusable dispensing actuator 108 that can be removed from a used bottle assembly 200 and inserted into a new prefilled bottle assembly 200. FIG. 4A illustrates an eccentric wheel 305a that is disengaged from the ball member 430. The ball member 430 is configured to engage with the eccentric wheel 305 when the dispensing actuator 108 is inserted into the dispensing actuator compartment 211 within the cup-shaped member 103.

Engagement and disengagement of the eccentric wheel 305 with the ball member 430 is indicated by an arrow 440. The eccentric wheel 305 may slide on a surface of the ball member 430 without interference which enables convenient replacement of the bottle assembly 200 by pulling the removable dispensing actuator 108 from the bottle assembly 200. The ball member 430 may have two or more functions. For example, the ball member 430 may deform the diaphragm 432 to the hemispherical shape 432a that has substantially the same radius of curvature as the cavity 435 such that sufficient suction is generated to prime the fluid path 410. In addition, the ball member 430 may allow interference-free sliding engagement and disengagement of the eccentric wheel 305 when the dispensing actuator 108 is pulled out of or inserted into the bottle assembly 200. This method enables convenient and cost-effective replacement of empty bottles with pre-filled bottles.

In the event that the dispensing system 100 is actuated and the dip tube 107 is not submerged in the fluid 105, there is a danger that the dispensing mechanism 400 will pull air into the fluid path 410 and its normal operation will be disrupted or the dispensing accuracy will be affected. To assure that the dispensing system 100 operates only in a substantially vertical orientation, the PCB 306 may include a 3-axis acceleration sensor or a tilt sensor that senses the orientation of the dispensing system 100 and prevents operation when the axis of the bottle 102 is inclined more than about 30 degrees away from the gravitational acceleration vector. In examples, the sensor may be made by or be similar to a Würth Elektronik sensor, part number 2533020201601. In examples, the tilt sensor may be configured to prevent operation of the dispensing actuator 108 when the bottle assembly 200 tilts beyond a predetermined angle with respect to an upright position of the bottle assembly 200.

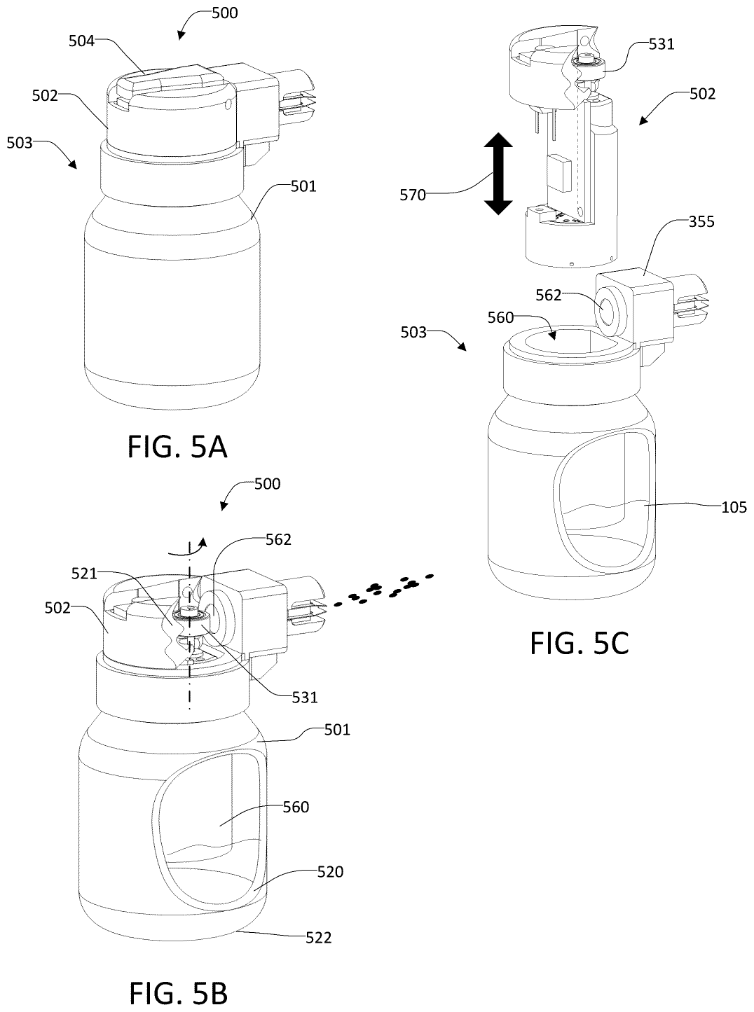

FIG. 5A illustrates a perspective view of a dispensing system (or device) 500 which includes a bottle assembly 503 and a removable dispensing actuator 502. The dispensing system 500 may include some or all of the functions discussed herein regarding the dispensing system 100. The dispensing actuator 502 may include a momentary switch 504 that activates a dispensing cycle.

FIG. 5B illustrates the dispensing system 500 including two cutouts. A first cutout 520 in a bottle 501 of the bottle assembly 503 shows a cup-shaped member 560 inserted into the bottle 501. A second cutout 521 in the dispensing actuator 502 shows an eccentric wheel 531 engaged with a ball member 562. The bottle 501 includes a bottom 522.

FIG. 5C illustrates the dispensing actuator 502 pulled out from a compartment 560 in the bottle assembly 503. The dispensing actuator 502 may be pulled from an empty bottle assembly and inserted into a new pre-filled bottle assembly. The ball member 562 enables sliding engagement and disengagement of the eccentric wheel 531 from the ball member 562 that activates the dispensing mechanism 400 as illustrated in FIG. 4A and FIG. 4B. The eccentric wheel 531 may be a part of the dispensing actuator 502 which can be removed from the bottle assembly 503 in the direction illustrated by arrows 570. The dispensing actuator 502 does not make any physical contact with the fluid 105 that is hermetically sealed in the bottle assembly 503 therefore replacement of the dispensing actuator 502 can be done without the risk of cross contamination.

Features of the examples of FIGS. 5A-5C may be utilized solely or in combination with any other example herein.

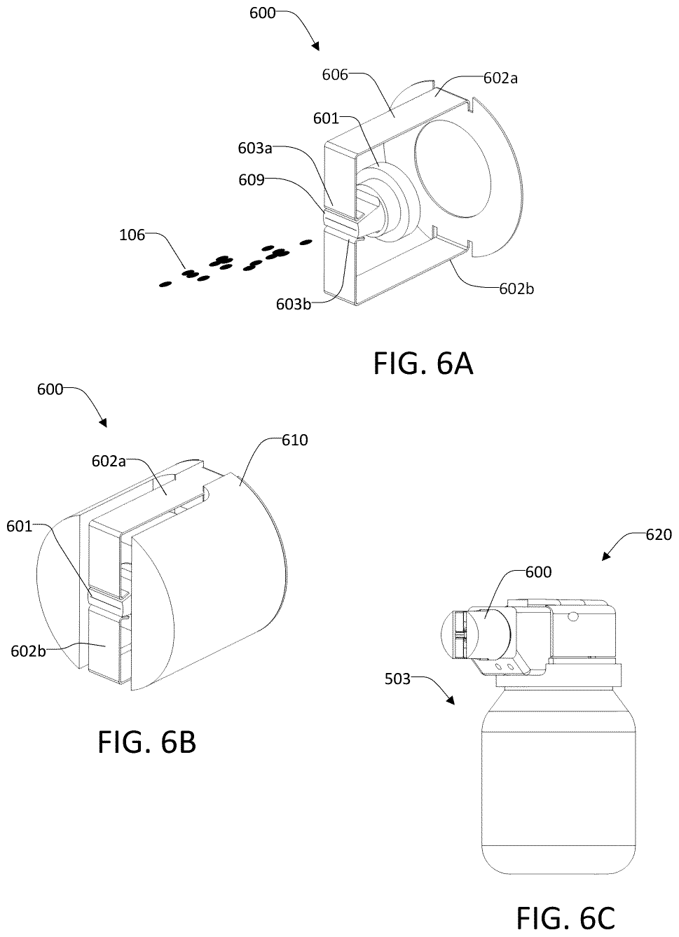

FIG. 6A illustrates a dispensing tip assembly 600. The dispensing tip assembly 600 may be positioned at the outlet conduit 438 (marked in FIG. 4B). In examples, the dispensing tip assembly 600 may be coupled to the nozzle block 355 (marked in FIGS. 3A and 5C). The dispensing mechanism 400 (marked in FIG. 4A and FIG. 4B) may generate rapid cycles of pressure fluctuation which include consecutive cycles of pressure and under-pressure with a one-way valve 601 (e.g., the dispensing tip 356 marked in FIG. 3A) opening during a pressure cycle to permit outflow and quickly closing as the following cycle of under-pressure begins. If the valve 601 does not respond quickly enough there is a danger that the upcoming cycle of under-pressure will pull contaminants into the bottle assembly 503 (or the bottle assembly 200). Preferably the valve 601 should be small and light weight such that the reflected inertia of moving lips 609 of the valve 601 will be minimal. In examples, the valve 601 may be or may be similar to a one-way valve model DU 047.001 SD duckbill valve manufactured by MiniValve International®.

In the preferred embodiment the closing force of the valve 601 is increased by two spring members including a first spring member 602a and a second spring member 602b with one spring member being on each side of the valve lips 609. A first free end 603a and a second free end 603b of the spring members 602a, 602b, respectively, may press against the valve lips 609 and apply a predetermined force that increases the minimum pressure that is required to open the valve 601 and allow outflow. The spring members 602a, 602b may be u-shaped beams and may be made of spring steel and typically may apply 5 grams (g) to 20 grams (g) of force against the valve lips 609. An advantage of the valve 601 being a duckbill (or horizontal) valve relative to conventional valves used for ocular fluid administration is that the valve 601 does not have a residual volume of fluid outside the outlet nozzle as shown, for example, in U.S. Pat. No. 9,238,532 and U.S. Pat. Pub. No. 2014/0336596. In some cases, the closing force of the duckbill valve lips 609 alone may be insufficient or inconsistent. However, the spring members 602a, 602b overcome this problem. The spring member 602a, 602b may increase the closing force of a duckbill valve.

FIG. 6B illustrates the dispensing tip assembly 600 further including a cylindrical member 610. The cylindrical member 610 may support the dispensing tip assembly 600 with the one-way valve 601 and the spring members 602a, 602b being assembled or positioned at least partially within the cylindrical member 610.

FIG. 6C illustrates the dispensing tip assembly 600 attached to a dispensing device 620 (e.g., any dispensing device or system as disclosed herein).

Features of the examples of FIGS. 6A-6C may be utilized solely or in combination with any other example herein.

In examples, a plurality of the bottles or bottle assemblies may be utilized that may be configured similarly as bottles or bottle assemblies disclosed herein. Actuators as disclosed herein may be configured to removably couple to another bottle assembly of the plurality of bottle assemblies when the fluid within the bottle assembly is depleted.

Exemplary embodiments of the methods/systems have been disclosed in an illustrative style. Accordingly, the terminology employed throughout should be read in a non-limiting manner. Although minor modifications to the teachings herein will occur to those well versed in the art, it shall be understood that what is intended to be circumscribed within the scope of the patent warranted hereon are all such embodiments that reasonably fall within the scope of the advancement to the art hereby contributed, and that that scope shall not be restricted, except in light of the appended claims and their equivalents.

What is claimed is:

1. A device for dispensing a predetermined amount of fluid to a surface of an eye, the device comprising:
a bottle assembly including:
a bottle enclosure having an opening and configured to hold the fluid,
a cup-shaped member that extends into the bottle enclosure through the opening of the bottle enclosure dividing an internal space of the bottle enclosure into a first compartment and a second compartment, the first compartment being a hermetically sealed fluid compartment defined by a volume between an internal surface of the bottle enclosure and the cup-shaped member, the second compartment being an internal volume inside the cup-shaped member, and
a dispensing mechanism coupled to the cup-shaped member and configured to dispense the predetermined amount of the fluid; and
an electric dispensing actuator configured to:
removably couple to the bottle assembly by sliding into the second compartment, and
engage and activate the dispensing mechanism to pull the fluid from the first compartment and dispense the fluid through a dispensing tip.

2. The device of claim 1, wherein the electric dispensing actuator comprises:
a housing including:
an eccentric wheel coupled to a motor,
a timer circuit coupled to the motor, and
one or more batteries coupled to the timer circuit and the motor; and
a momentary switch configured to initiate rotation of the eccentric wheel by the motor for a period of 50-100 milliseconds (msec) when actuated by a user.

3. The device of claim 2, wherein the dispensing mechanism comprises:
a solid structure having a hemispherical cavity sealed by a diaphragm;
a ball member concentrically aligned to the hemispherical cavity and tangentially engaged with the diaphragm and configured to cyclically displace the diaphragm within the hemispherical cavity;
an inlet conduit located within the hemispherical cavity and configured to be in fluid communication with the first compartment; and
an outlet conduit located within the hemispherical cavity and configured to be in fluid communication with the dispensing tip, the dispensing tip being a one-way valve.

4. The device of claim 3, wherein:
the diaphragm is flat and covers the hemispherical cavity; and
the electric dispensing actuator activates the dispensing mechanism by turning the eccentric wheel to cyclically oscillate the ball member and the diaphragm toward a hemispherical surface of the hemispherical cavity.

5. The device of claim 3, wherein the ball member is configured to engage with the eccentric wheel when the electric dispensing actuator is inserted into the second compartment within the cup-shaped member.

6. The device of claim 1, wherein the dispensing mechanism includes a cavity and a fluid inlet conduit that extends from the cavity to the first compartment and an outlet conduit that extends to the dispensing tip.

7. The device of claim 6, wherein the dispensing tip includes a one-way valve.

8. The device of claim 1, wherein the dispensing tip includes a duckbill valve and one or two spring members that increase a closing force of the duckbill valve.

9. The device of claim 1, wherein the bottle enclosure and the cup-shaped member each have a cylindrical shape.

10. The device of claim 1, wherein the electric dispensing actuator includes a tilt sensor configured to prevent operation of the electric dispensing actuator when the bottle assembly tilts beyond a predetermined angle with respect to an upright position of the bottle assembly.

11. The device of claim 1, wherein a volume of the bottle enclosure is 10-30 milliliters (mL).

12. The device of claim 1, wherein the predetermined amount of the fluid that is dispensed upon each actuation through the dispensing tip is 5-20 microliters (μL).

13. The device of claim 1, wherein the predetermined amount of the fluid that is dispensed upon each actuation through the dispensing tip is less than 30 microliters (μL).

14. A dispensing mechanism for ophthalmic delivery of fluid medicament to a surface of an eye, comprising:
a hemispherical cavity sealed by a diaphragm and including:
an inlet conduit in fluid communication with a fluid compartment, and
an outlet conduit in fluid communication with a one-way valve; and
a ball member that is concentrically aligned to the hemispherical cavity and tangentially engaged with the diaphragm and configured to cyclically displace the diaphragm into the hemispherical cavity.

15. A dispensing system for dispensing a predetermined amount of fluid to a surface of an eye, the system comprising:
a plurality of bottle assemblies each including:
a bottle enclosure having an opening,
a cup-shaped member configured to extend into the bottle enclosure through the opening of the bottle enclosure to form a hermetically sealed fluid compartment within the bottle enclosure for holding the fluid, and
a dispensing mechanism coupled to the cup-shaped member and configured to dispense the predetermined amount of the fluid; and
an electric dispensing actuator configured to:
removably couple to a bottle assembly of the plurality of bottle assemblies by sliding into the cup-shaped member,
engage and activate the dispensing mechanism to pull the fluid from the hermetically sealed fluid compartment and dispense the fluid through a dispensing tip, and
removably couple to another bottle assembly of the plurality of bottle assemblies when the fluid within the bottle assembly is depleted.
